(19) United States
(12) Patent Application Publication
(10) Pub. No.: US 2026/0146218 A1
(43) Pub. Date: May 28, 2026

(54) Bioreactor for Brine Denitrification and Methods of Use

(71) Applicants: University of Maryland, Baltimore, Baltimore, MD (US); University of Maryland, College Park, College Park, MD (US)

(72) Inventors: Shiladitya DasSarma, Ellicott City, MD (US); Priya DasSharma, Ellicott City, MD (US); Leonardo Marcello Soto Chumpitaz, Elkridge, MD (US)

(73) Assignees: University of Maryland, Baltimore, Baltimore, MD (US); University of Maryland, College Park, College Park, MD (US)

(21) Appl. No.: 19/351,811

(22) Filed: Oct. 7, 2025

Related U.S. Application Data
(60) Provisional application No. 63/704,111, filed on Oct. 7, 2024.

(51) Int. Cl. C12M 1/107

(52) U.S. Cl. CPC C12M 21/04

(57) ABSTRACT

The present invention is a bioreactor, for example, an Archael bioreactor, used in processes to denitrify brine. Halophilic denitrifying microbes are contained in an aqueous medium within the bioreactor that enables anaerobic respiration in the brine to produce nitrogen gas as an end product. The denitrified brine may be used to refresh a spent chromatography column in a water purification process.

### CROSS-REFERENCE TO RELATED APPLICATIONS

This non-provisional patent application claims benefit of priority under 35 U.S.C. § 119(e) of provisional application U.S. Ser. No. 63/704,111, filed Oct. 7, 2024, the entirety of which is hereby incorporated by reference.

### BACKGROUND OF THE INVENTION

### Field of the Invention

The present invention relates to the fields of microbiology and bioremediation. More particularly, the present invention relates to methods for removing nitrates and nitrites from brine using halophilic denitrifying microorganisms.

### Description of the Related Art

Nitrogen is an abundant and important element for many geological and biological processes on Earth and the global nitrogen cycle has been well-studied (1, 2). A key intermediate in the nitrogen cycle, nitrate, is classified as a toxic pollutant and occurs naturally in groundwater. It is also commonly found in manure runoff (which varies by animal type), from synthetic fertilizers on cropland and pastures and as discharge from animal feeding efforts (3). Nitrate also leaches from septic tanks and sewage, and may contaminate both surface and groundwater. Additionally, nitrate is also released as a result of wastewater treatment plant and septic system discharges, fossil fuel combustion, urban runoff, and landfill leachate (4). With predicted increase in fertilizer usage due to changing agricultural conditions, there is an urgent need for developing effective denitrification processes.

Self-supplied drinking water systems (98% of which depend on groundwater wells), are not federally regulated and depend on individual well-owners to test and treat for nitrate and other pollutants. Many municipal drinking water supplies have also been found to exceed permitted levels of nitrate (5, 6). Fertilizer often reaches both surface and groundwater systems through farm or urban and suburban runoff or infiltration (7).

Additionally, elevated nitrate loads have been identified in downstream water bodies and coastal systems including, e.g., the Chesapeake Bay, and Puget Sound, leading to hypoxia (dissolved oxygen concentrations <2 mg/L) and subsequent eutrophication (8). This results in overgrowth of cyanotoxin-producing cyanobacteria that cause harm to human health, cause fish kills, and death to mammals and birds leading to an overall decrease in biodiversity (9). Presence of 3 mg/L (3 ppm) of nitrate generally indicates ground water contamination (10), with nitrate concentrations above 1 mg/L (1 ppm) considered to be a concern for use as drinking water (11, 12). The EPA determined that nitrate levels in drinking water should not exceed 10 mg/L (10 ppm) (13, 14).

In the United States, a growing number of wells require nitrate remediation at a rate of over 150,000 gallons per minute of purified water. Currently, the conventional method is to use anion exchange resin (IER) chromatography to remove the nitrate. This general process is also used to remove many other anionic species, including nitrite, as well as arsenic, bicarbonate, fluoride, phosphate, sulfate, and sulfite. The exhausted anion exchange systems are generally regenerated with strong sodium chloride (brine) solutions consisting of chloride anions, which reattach to the resin beads and release the exchanged anions during a backwash process. This results in purified drinking water, but also results in a by-product: nitrate-contaminated brine. Nitrate concentrations of 1,000 mg/1 (1,000 ppm) are common in the resulting waste brine and must be subsequently treated or disposed of. This contaminated brine is both expensive and difficult to dispose of and hazardous to both the environment and public health. Estimates are that 1,200 gallons per minute (or over 2 billion liters per year) of nitrate-contaminated waste brine is generated nationally.

Although biological denitrification is an effective treatment process for the removal of nitrate from contaminated water, the presence of strong brines represents a significant challenge to most microbial activity. No current large-scale process for nitrate removal from strong brines has been commercialized. Thus, there is an unmet need for methods for large scale remediation of these nitrate-contaminated brines.

The prior art is deficient in methods for bioremediation of nitrates in brines. Particularly, the art is deficient in bioreactors utilizing Haloarchaea to remove nitrates from brines and in methods for bioremediating nitrate-contaminated brine. The present invention fulfills this long-standing need and desire in the art.

### SUMMARY OF THE INVENTION

The present invention is directed to a device to denitrify a nitrate-contaminated brine. The device has a vessel with an inlet to fluidly receive the nitrate-contaminated brine therein, a first outlet to fluidly remove the denitrified brine therefrom and a second outlet through which to release nitrogen gas. An aqueous medium is contained within the vessel to support anaerobic microbial growth and halophilic denitrifying microbes are dispersed within the aqueous medium. The present invention is directed to a related device further comprising a reservoir for the denitrified brine in fluid connection with the first outlet in the vessel.

The present invention is further directed to a method for removing nitrates from brine. In this method, the brine flows through the inlet into the device described herein. The brine is denitrified via anaerobic respiration of the halophilic denitrifying microbes dispersed within the aqueous medium, thereby producing nitrogen gas. The denitrified brine flows through the first outlet and the nitrogen gas is released through the second outlet. The present invention is directed to a related method further comprising storing the denitrified brine in a reservoir for use in a water purification system.

The present invention is directed further to a bioremediation device to remove nitrate contaminants from brine. The device is an Archaeal bioreactor that comprises an inlet to fluidly receive the brine therein, a first outlet to fluidly remove denitrified brine therefrom, a second outlet through which to release nitrogen gas, an aqueous medium effective to support anaerobic growth of an Haloarchaea contained within the Archaeal bioreactor, and a variant of Haloferax mediterranei A1 dispersed within the aqueous medium. A brine reservoir is fluidly connected to the first outlet.

The present invention is directed further still to a method for bioremediating nitrate-contaminated brine during a water purification process. In this method, the nitrate-contaminated brine from a spent chromatography column produced during the water purification process is received through the inlet into the Archaeal bioreactor described herein. The nitrate-contaminated brine is denitrified via anaerobic respiration by the variant of Haloferax mediterranei A1 thereby producing nitrogen gas. The denitrified brine is flowed through the first outlet into the brine reservoir and the nitrogen gas is released through the second outlet into the atmosphere. The spent chromatography column is regenerated with the denitrified brine in the brine reservoir for reuse in the water purification process. The present invention is directed to a related method that further comprises repeating the method steps at least once until the water purification process is complete.

Other and further aspects, features, benefits, and advantages of the present invention will be apparent from the following description of the presently preferred embodiments of the invention given for the purpose of disclosure.

### BRIEF DESCRIPTION OF THE DRAWINGS

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIG. 1 shows nitrate removal from drinking water using IER chromatography followed by removal and replacement of spent column. Nitrate-contaminated brine is currently disposed of but the new technology may be used for denitrification by NDA4 in an Archaeal bioreactor after backwashing.

FIG. 2 shows the growth of six Haloarchaeal strains: 1. Haloferax mediterranei (Hme), 2. Natrinema pallidum NMX16-2 (NMX16-2), 3. Natrinema pallidum BOL6 -1 (BOL6-1), 4. Haloarcula marismortui (Hma), 5. Haloferax volcanii (Hvo), and 6) Halorubrum lacusprofundi (Hla).

FIG. 3 shows a comparison of anaerobic growth of HME isolates under denitrifying conditions. Cultures were grown in Hme minimal media with or without glutamate, bubbled with N₂ for 30 mins 42° C. with shaking, light at 100 rpm.

FIG. 4 shows that superior growth with nitrate was observed for strain NDA4 (orange) over parent strain NDA1 (green). No growth was obtained anaerobically without nitrate (data not shown).

FIG. 5 shows testing of alternate carbon sources: Comparing NDA4 growth with various carbon sources (time in days plotted on x-axis and OD 600 nm plotted on y-axis). Growth results using different media without glutamate or sucrose, testing alternate carbon sources (King corn syrup or soluble starch or two types of corn syrup: King or Karo or soluble starch) anaerobically under illuminated at 42° C. with shaking at 110 rpm.

FIGS. 6A-6B are hydropathy plots comparing wildtype Hme HFX_2180 (FIG. 6A) and NDA4_2180 (FIG. 6B) plotted using the Pepwindow program and indicating where the two proteins diverge.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention. It is contemplated that any composition, component or method described herein can be implemented with respect to any other composition, component or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein “another” or “other” may mean at least a second or more of the same or different claim element or components thereof.

As used herein, the terms “comprise” and “comprising” are used in the inclusive, open sense, meaning that additional elements may be included.

As used herein, the terms “consist of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure. In a non-limiting example, the nitrates limit of about 2,300 ppm encompasses 2,070 ppm to 2530 ppm.

As used herein, the ordinal adjectives “first” and “second” unless otherwise specified are used to describe a common object, merely indicate that different instances of like objects are being referred to, and are not intended to imply that the objects so described must be in a given sequence, either temporally, spatially, in ranking, or in any other manner.

As used herein the terms “bioreactor”, “bioremediation device”, and “Archaeal bioreactor” are interchangeable.

In one embodiment of the present invention, there is provided a device to denitrify a nitrate-contaminated brine, comprising a vessel with an inlet to fluidly receive the nitrate-contaminated brine therein, a first outlet to fluidly remove the denitrified brine therefrom and a second outlet through which to release nitrogen gas; an aqueous medium contained within the vessel to support anaerobic microbial growth; and halophilic denitrifying microbes dispersed within the aqueous medium.

In both embodiments, the halophilic denitrifying microbe may be a Haloarchaeaon. Particularly, the Haloarchaeaon is Haloferax mediterranei A4 (NDA4). Also the Haloferax mediterranei A4 (NDA4) is growable in brine containing about 7,000 ppm of nitrate. In addition, the vessel may be a bioreactor.

In another embodiment of the present invention, there is provided a method for removing nitrates from brine, comprising flowing the brine through the inlet into the device as described supra; denitrifying the brine via anaerobic respiration of the halophilic denitrifying microbes dispersed within the aqueous medium, thereby producing nitrogen gas; flowing denitrified brine through the first outlet; and releasing the nitrogen gas through the second outlet. Further to this embodiment, the method comprises storing the denitrified brine in a reservoir for use in a water purification system. In aspects of this further embodiment, the water may be ground water, surface water, wastewater, or well water.

In both embodiments and aspects thereof, the releasing step may comprise releasing the nitrogen gas into the atmosphere. The brine may preferably contain about 12% salt to about 17% salt. In addition, the halophilic denitrifying microbes are Haloferax mediterranei A4 (NDA4), where the denitrifying step may comprise removing about 2,300 ppm nitrates from the brine at a rate of 56 ppm/day to 71 ppm/day.

In yet another embodiment of the present invention, there is provided a bioremediation device to remove nitrate contaminants from brine, comprising an Archaeal bioreactor comprising an inlet to fluidly receive the brine therein, a first outlet to fluidly remove denitrified brine therefrom; a second outlet through which to release nitrogen gas; an aqueous medium effective to support anaerobic growth of an Haloarchaea contained within the Archaeal bioreactor; and a variant of Haloferax mediterranei A1 dispersed within the aqueous medium; and a brine reservoir fluidly connected to the first outlet. In this embodiment, the variant of Haloferax mediterranei A1 is Haloferax mediterranei (NDA4), where the NDA4 is growable in brine containing about 7,000 ppm of nitrate.

In yet another embodiment of the present invention, there is provided a method for bioremediating nitrate-contaminated brine during a water purification process, comprising method for bioremediating nitrate-contaminated brine during a water purification process, comprising a) receiving, through the inlet into the Archaeal bioreactor as described supra, the nitrate-contaminated brine from a spent chromatography column produced during the water purification process; b) denitrifying the nitrate-contaminated brine via anaerobic respiration by the variant of Haloferax mediterranei A1 thereby producing nitrogen gas; c) flowing denitrified brine through the first outlet into the brine reservoir; d) releasing the nitrogen gas through the second outlet into the atmosphere; and e) regenerating the spent chromatography column with the denitrified brine in the brine reservoir for reuse in the water purification process. Further to this embodiment, the method comprises repeating steps a) to e) at least once until the water purification process is complete.

In both embodiments, the variant of Haloferax mediterranei A1 is Haloferax mediterranei A4 (NDA4). In both embodiments, the brine may contain about 12% salt to about 17% salt. The halophilic denitrifying microbes are preferably Haloferax mediterranei A4 (NDA4), where the denitrifying step comprising removing about 2,300 ppm nitrates from the brine at a rate of 56 ppm/day to 71 ppm/day. In aspects thereof, the water in the water purification process may be ground water, surface water, wastewater, or well water.

Provided herein are devices, for example, a bioreactor, a bioremediation device or an Archaeal bioreactor, effective to remove nitrate contaminants from brine. Processes using these devices utilize halophilic microbes that are able to withstand high concentrations of brine to denitrify the nitrate brine, producing an inert nitrogen gas as the end-product releasable into the environment.

The bioreactor contains a variant of the halophilic Archaeon Haloferax mediterranei A1 (HME-A 1). The variant strain, Haloferax mediterranei A4 (NDA4) is non-pathogenic and non-toxic and grows optimally in salt concentrations found, for example, in the spent brine from IER chromatography. The sequencing of the NDA4 strain showed a key mutation that explains the enhanced phenotype of denitrification.

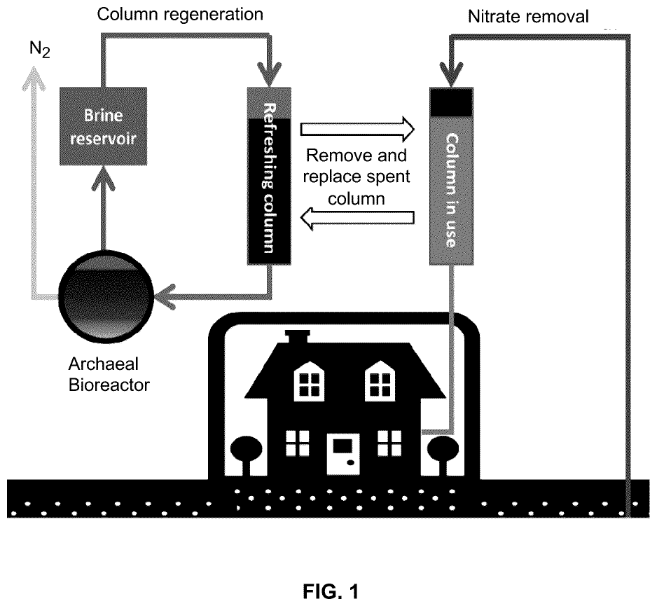

Also provided are methods for bioremediation of nitrates from water, for example, but not limited to, drinking or well water, ground water, surface water and wastewater. In a non-limiting example of bioremediation of nitrate contaminated water (FIG. 1), nitrate is initially removed through affinity chromatography using ion exchange resins, followed by column regeneration with concentrated sodium chloride solution. The resulting brine, containing high levels of nitrate anions, is treated using the NDA4 halophilic microbes to convert nitrates into harmless nitrogen gas. The denitrified brine may then be used for column regeneration. This method is an efficient, scalable solution for nitrate removal. long-term ecological and health benefits as a toxic pollutant, nitrate, is converted into an innocuous gas, N₂, a natural component of air.

The following examples are given for the purpose of illustrating various embodiments of the invention and are not meant to limit the present invention in any fashion.

### EXAMPLE 1

### Halophilic Microorganisms Tested

Halophilic microorganisms to denitrify brine containing high concentrations of nitrate. Halophilic microbes are an evolutionary distinct group of organisms that are uniquely able to survive in and exploit hypersaline environments. These microorganisms thrive in environments with high concentrations of salt—from brines to inclusions within salt crystals (15). Archaeal halophilic microbes are a safe, non-toxic, and non-pathogenic class of organisms, which are regularly consumed in oriental sauces and utilized for teaching microbiology.

Several strains of halophilic microorganisms have been reported to carry out denitrification, including the archaea (haloarchaea), such as Haloferax mediterranei and Haloferax volcanii. These archaea are distinguished by their ability to grow in very high salinities and their growth properties have also been characterized in some detail. For H. mediterranei, growth using either nitrate as the sole electron acceptor has been demonstrated in minimal media and its gene cluster containing the nitrate reductase (nar) genes cloned and characterized.

### EXAMPLE 2

### Formulations of Brines for Use in Backwashing

In order to develop media formulations for growth of denitrifying microbes that can function at the high salt concentrations produced by the IER chromatography process, two main hurdles have to be passed: (1) Finding a strain that grows at the high salinity and also denitrifies, and (2) a media composition that will be inexpensive and does not interfere with nitrate removal from spent brine produced by column regeneration.

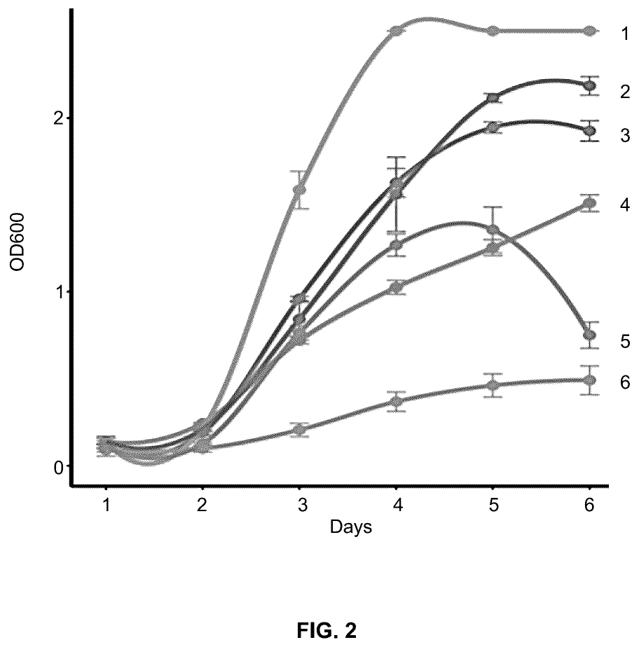

Six target halophilic strains were selected to examine for their ability to denitrify (bioremediate) contaminated brines in an environmentally friendly manner (FIG. 2). These were: Halorubrum lacusprofundi (Hla), Haloarcula marismortui (Hma); and Haloferax mediterranei (Hme); Haloferax volcanii (Hvo) gifted by the American Type Culture Collection (ATCC) and Natrinema pallidum BOL6-1 (BOL6-1); and Natrinema pallidum NMX16-2 (NMX16-2, also known as WIPP16-2) isolated from Bolivia and New Mexico, USA by the DasSarma laboratory (16).

Three of the Haloarchaea [Haloferax mediterranei (Hme); Natrinema pallidum NMX16-2 (NMX16-2); and Natrinema pallidum BOL6-1 (BOL6-1)] were able to grow anaerobically in the presence of the nitrates, and denitrify the aqueous medium to reduce the levels of nitrates from the original level. They were subcultured under selective pressure. The most effective strain, HME-A1 was screened by subculturing to select for even more efficient denitrifying strains (see below). Strain NDA4 was found to be the most effective denitrifier. It was able to fully denitrify an aqueous medium containing up to 1,200 ppm nitrates in aqueous media containing from 12% to 17% salt (similar to the 15% NaCl general brine concentration from IER chromatography).

### EXAMPLE 3

### Testing for Growth in the Presence of Nitrate

In order to develop a method for bioremediating nitrate-contaminated brines, multiple denitrifying microbes were tested. Culturing media for these organisms typically contain between 2.5-4.5 M NaC1(15 % and 20% total salinities). Cultures were grown under aerobic and anaerobic conditions in the presence or absence of nitrate. For anaerobic growth, the medium was sparged with nitrogen and the cultivation vessel containing the medium was sealed with a rubber stopper, and the electron acceptor added from a sterile stock solution prior to sparging. Cultivation vessels were incubated with shaking (at 100-200 rpm) at 42° C. with illumination.

Growth was monitored spectrophotometrically by measuring the optical density in a Klett meter (Klett Manufacturing Co.). Growth was obtained for all strains tested aerobically without nitrate in their optimal media. However, only one strain, HME-1 grew well anaerobically with nitrate (100 or 1000 ppm), while little or no growth was obtained anaerobically without nitrate, confirming the ability of this organism to grow by dissimilatory nitrate reduction (data not shown). Liberation of nitrogen gas was observed, consistent with conversion of nitrate to gaseous dinitrogen during denitrification.

In order to optimize the concentration of salts in the growth medium for HME-1, media was tested containing 10%, 14%, and 16% total dissolved salts (including sulfate). It was found that while a concentration of 16% salts produces the best growth rate anaerobically in the presence of 1000 ppm nitrate, 14% also allowed good growth, (data not shown). Therefore, there is a relatively broad salt optimum for this strain. However, the lowest concentration of salts tested, 10%, resulted in poor growth.

### EXAMPLE 4

### Nitrate Content Determination in 14% and 16% Salts Cultures

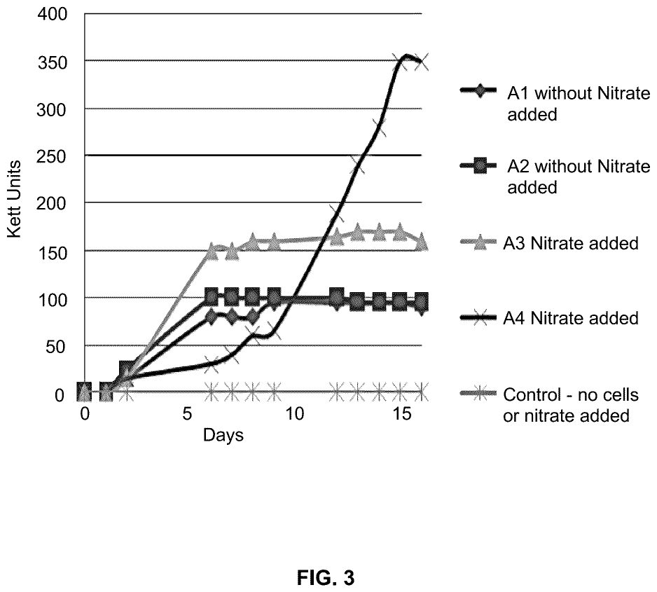

The nitrate content of the culture medium was tested following removal of cells by filtration. Nitrate content was determined either using a test kit from Industrial Test Systems Inc or sent out for testing. Waste brines were tested for residual nitrate content. Significant reduction in nitrate content was observed for cultures grown in the presence of both 14% and 16% dissolved salts with nitrate. Interestingly, during these studies, one isolate was discovered that displayed enhanced growth compared to the parent HME-1 strain (FIG. 3). This variant was named HME-A4 (now known as NDA4). The enhanced growth of this strain anaerobically and in the presence of nitrate suggested that it would serve as an improved strain for denitrification.

Further experimentation resulted in the determination that NDA4 cannot only fully denitrify brines containing 1,200 ppm nitrate but also shows promise in denitrifying solutions containing 2,300 ppm at a rate of 56-71 ppm/day. The strain is able to grow in the presence of 7000 ppm nitrate (data not shown).

### EXAMPLE 5

### Nitrate Content Determination in Cultures Without Glutamate

In order to improve the laboratory media composition, we started with removal of glutamate as a major carbon and energy source, since this is expected to bind to the anion exchange column and thereby prevent full regeneration. Glutamate was substituted with various sugar sources and starch. Starch was found to serve as a good carbon source, albeit not as good as with the addition of both glutamate and sugar.

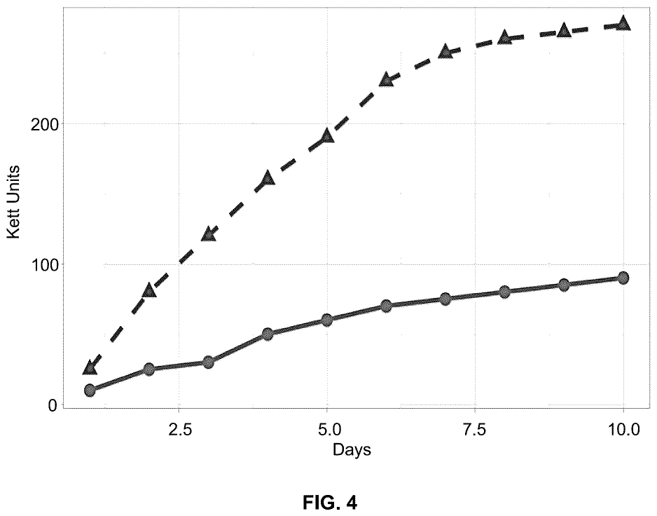

The nitrate content of the culture medium lacking glutamate following removal of cells was tested. It was observed that a subculture of HME A-1 with denitrification capabilities developed enhanced growth capability in nitrate under anaerobic conditions. We tested the ability of this isolate from this culture, named, NDA-4, for growth in increasing concentrations of nitrate under anaerobic conditions and denitrification capabilities. Compared to the parent, NDA4 displayed superior growth (FIG. 4) and denitrification, with excellent growth at 1,000 ppm nitrate concentration. The strain was shown to be able to grow using a variety of carbon sources, including glutamate, high fructose corn syrup, starch, glucose and sucrose.

It was demonstrated that the material which would be toxic to most microorganisms can be utilized to generate energy for growth by this microorganism. The optimum salt concentration for the growth of this denitrifying microorganism is 14-16%, which is also the concentration of salt found in the nitrate-contaminated IER spent brine. It was determined that the nitrate was converted into N₂, a normal and completely non-toxic component of air, which solves the major current problem of IER nitrate-contaminated brine disposal. Also, the growth of the organism using sugar was shown to be a good carbon source, replacing glutamate (normally utilized in laboratory culturing).

Most significantly, a mutant variant of HME-A1, named NDA4, was isolated and showed enhanced denitrification capabilities, with most of the growth and denitrification occurring within 5 days. The nitrate concentrations in these cultures were reduced from 1,200 ppm, which is typical for the column discharge (and even higher concentrations), to zero demonstrating the efficacy needed for a process that would be available for reuse in IER columns for additional rounds of denitrification or discharged with minimal dilution.

### EXAMPLE 6

### Optimizing Media Composition

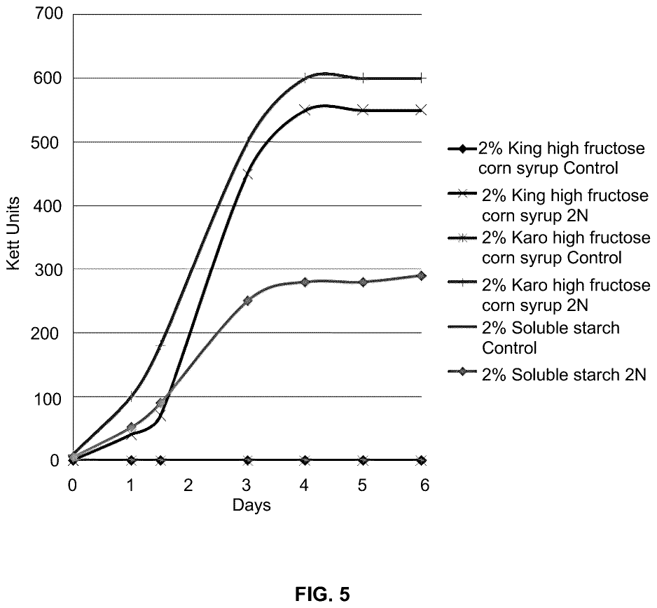

In addition, various growth conditions and media compositions were tested for nutrient content and cost efficiency optimization (FIG. 5). Eight formulations of media (Haloferax mediterranei Medium (HmeMed), Haloferax mediterranei Minimal Medium 1 Hme-A, Haloferax mediterranei Minimal Medium 2 (Hme-B), BH Minimal Media (BH), BH without Glutamate (BH-G), PD-CS a and b and PD-S) were tested to determine optimized composition for growth. Specifically, two types of commercial corn syrup (King or Caro) were tested. Additionally, starch was tested as a carbon source (FIG. 5).

Optical density was recorded using either a Klett meter or Spectronic Photometer. Media formulations used (per liter): Haloferax mediterranei Complete Medium [HmeMed] (195 g NaCl, 34.6 g MgCl2 6H20, 49.4 g MgSO4.7H2O, 0.92 g CaCl2 6H20, 0.5 g KCl, 0.17 g NaHCO3, 0.58 g NaBr, 5 g Yeast Extract, pH 7.2); Haloferax mediterranei Minimal Medium 1 (17) [Hme-A] (156 g NaCl, 13 g MgCl2 6H20, 20 g MgSO4.7H2O, 1 g CaCl2 6H20, 4 g KCl, 0.2 g NaHCO3, 0.5 g NaBr, 2 g NH4Cl, 0.005 g FeCl3 6H20, 0.5 g KH2PO4, 10 g C6H12O6, pH 7.2); Haloferax mediterranei Minimal Medium 2 (17) [Hme-B] (160 g NaCl, 20 g MgSO4.7H2O, 4 g KCl, 0.2 g KH2PO4, 20 g C5H8NNaO4.xH2O, 10 g C12H22O11, pH 7.2); BH Minimal Media [BH]; (160 g NaCl, 13 g 20 g MgSO4.7H2O, 4 g KCl, 20 g C5H8NNaO4.xH2O, 10 gC12H22O11, 0.3 g K2HPO4, pH 7.2); BH without Glutamate [BH-G] (160 g NaCl, 13 g 20 g MgSO4.7H2O, 4 g KCl, 10 g C12H22O11, 0.3 g K2HPO4, pH 7.2); PD-CS (using commercial High Fructose Corn Syrup (b): King syrup, golden composed of: corn syrup, high fructose corn syrup, refiners syrup, water, potassium sorbate (preservative), and citric acid or Caro Syrup (b)) (160 g NaCl, 13 g 20 g MgSO4.7H2O, 4 g KCl, 10 g C12H22O11, 10 ml v/v high fructose corn syrup, pH 7.2). PD-S (160 g NaCl, 13 g 20 g MgSO4.7H2O, 4 g KCl, 10 gC12H22O11, 2% soluble starch, pH 7.2). Media were also prepared using varying amounts of salts (2.5-4.5 M NaCl).

Karo Dark Corn Syrup contains: dark corn syrup, refiners syrup, caramel flavor, salt, sodium benzoate (used to protect quality), and caramel color. King syrup golden contains: corn syrup, high fructose corn syrup, refiners syrup, water, potassium sorbate (preservative), and citric acid.

### EXAMPLE 7

### Sequence Analysis of NDA4 Strain

The NDA4 strain genome was subsequently sequenced using PacBio sequencing in collaboration with New England Biolabs (Ipswich, MA). Sequencing resulted in an assembled genome with a chromosome of ˜3.9 Mb (80× coverage) and 3 plasmids of 504 kb (62× coverage), 322 kb (45× coverage), and 132 kb (31× coverage). After gene prediction was conducted using GeneMarkHMM, the resulting proteome was compared to the proteome of the closest relative, Haloferax mediterranei strain ATCC 33500 (18, 19), using BlastP analysis.

The sequence analysis identified a variant cytochrome oxidase gene, HFX_21080, with a frameshift mutation. The cytochrome oxidase is likely used when O₂ levels are low, similar to CydA and B, in Halobacterium sp. NRC-1 (20). The cydA and cydB genes encode cytochrome C and quinol oxidase polypeptide I, belonging to pfam00115, and containing the CDD:459678 Heme-copper oxidase subunit I. Heme-copper oxidases are known to be transmembrane protein complexes in the respiratory chains of prokaryotes and mitochondria. A similar function is consequently predicted for HFX_21080.

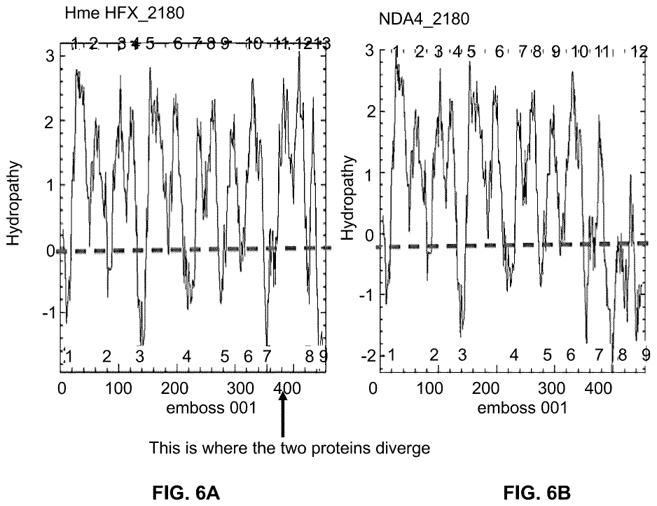

The frameshift resulted in differences that are apparent from amino acid residue 383 to 455 in HFX_21080 cytochrome oxidase. As a result, based on hydropathy analysis using the Pepwindow program (21), there are 13 helical regions predicted for the WT, and 12 for the NDA4 2180 protein. Based on the regions indicated by the arrows, the mutation will impact the function of this important protein based on the observed differences in protein length and hydropathy characteristics (FIGS. 6A-6B). This frameshift mutation would result in a non-functional cytochrome oxidase in the NDA4 strain, which is not able to use low concentrations of O₂ for aerobic growth. Consequently, the NDA4 microbe shifts using nitrate as the terminal electron acceptor at an earlier growth stage, which results in the observed improved growth anaerobically when nitrate is available.

### REFERENCES

1. Zhang, et al. Chemical Reviews. 120(12):5308-5351, 2020.
2. Kanehisa, et al., Nucleic Acids Research, 53(D 1):D672-D677, 2025.
3. EPA, Estimated Animal Agriculture Nitrogen and Phosphorus from Manure,
epa.gov/nutrientpollution/estimated-animal-agriculture-nitrogen-and-phosphorus-manure, last accessed Jul. 22, 2025.
4. EPA, Nutrient Indicators Dataset, epa.gov/nutrientpollution/nutrient-indicators-dataset, last accessed Jul. 22, 2025.
5. Wang, et al. Environ Sci Technol. 57(48):19395-19406, 2023.
6. Ward, et al. Int J Environ Res Public Health, 15(7):1557, 2018.
7. United States Geological Survey (USGA). 1984. Water-Supply Paper 2275. pubs.usgs.gov/wsp/2275/report, last accessed Jul. 22, 2025.
8. EPA, Estimated Total Nitrogen and Total Phosphorus Loads and Yields Generated within States, epa.gov/nutrientpollution/estimated-total-nitrogen-and-total-phosphorus-loads-and-yields-generated-within, last accessed Jul. 22, 2025.
9. EPA, Lakes Presenting Risk for Exposure to Harmful Algal Toxins, epa.gov/nutrientpollution/lakes-presenting-risk-exposure-harmful-algal-toxins, last accessed Jul. 22, 2025.
10. Madison, R. J. and Brunett, J. O. 1985. Overview of the occurrence of nitrate in ground water of the United States, in National Water Summary 1984-Hydrologic Events, Selected Water-Quality Trends, and Ground-Water Resources: U.S. Geological Survey Water-Supply Paper 2275, pp. 93-105, 1985.
11. Dubrovsky, et al. The quality of our Nation's waters—Nutrients in the Nation's streams and groundwater, 1992-2004: U.S. Geological Survey Circular 1350, 2010.
12. Chaudhary, et al. Water Conserv Sci Eng 10, 33, 2025.
13. EPA, National Primary Drinking Water Regulations, epa.gov/ground-water-and-drinking-water/national-primary-drinking-water-regulations, last accessed Jul. 22, 2025.
14. U.S. Environmental Protection Agency: Basic Information about Nitrate in Drinking Water, water.epa.gov/dring/contaminants/basicinformation/nitrate.cfm, last accessed Jul. 22, 2025.
15. DasSarma, S., and DasSarma, P., Halophiles, In book eLS:1-13, 2017.
16. DasSarma, et al. Microbiology Resource Announcements 8: e00810-19, 2019.
17. DasSarma, S., and Fleischmann, E. M. Media for Halophiles. In Halophiles. Eds. S. DasSarma and E. M. Fleischmann. Archaea. A Laboratory Manual. Cold Spring Harbor Laboratory Press, New York, 1995.
18. DasSarma, et al. Microbiol. Resour. Announc. 8: e00577-19, 2019.
19. DasSarma, et al. Saline Systems 6:12, 2010.
20. DasSarma, et al. J. Bacteriol. 194:5520-5537, 2012.
21. Madeira, et al. Nucleic Acids Research, 52(W1):W521-W525, Jul 2024.
22. Dodds, et al. Environmental Science and Technology. 43(1):12-19, 2009.
23. Wurtsbaugh, et al. WIREs Water, 6:e1373, 2019.
24. Davidson, et al. Journal of Environmental Management, 146:206-216, 2014.

What is claimed is:

1. A device to denitrify a nitrate-contaminated brine, comprising:
a vessel with an inlet to fluidly receive the nitrate-contaminated brine therein, a first outlet to fluidly remove the denitrified brine therefrom and a second outlet through which to release nitrogen gas;
an aqueous medium contained within the vessel to support anaerobic microbial growth; and
halophilic denitrifying microbes dispersed within the aqueous medium.

2. The device of claim 1, further comprising a reservoir for the denitrified brine in fluid connection with the first outlet in the vessel.

3. The device of claim 1, wherein the halophilic denitrifying microbe is a Haloarchaea.

4. The device of claim 3, wherein the Haloarchaea is Haloferax mediterranei A4 (NDA4).

5. The device of claim 3, said Haloferax mediterranei A4 (NDA4) is growable in brine containing about 7,000 ppm of nitrate.

6. The device of claim 1, wherein said vessel is a bioreactor.

7. A method for removing nitrates from brine, comprising:
flowing the brine through the inlet into the device of claim 1;
denitrifying the brine via anaerobic respiration of the halophilic denitrifying microbes dispersed within the aqueous medium, thereby producing nitrogen gas;
flowing denitrified brine through the first outlet; and
releasing the nitrogen gas through the second outlet.

8. The method of claim 7, further comprising storing the denitrified brine in a reservoir for use in a water purification system.

9. The method of claim 8, wherein the water is ground water, surface water, wastewater, or well water.

10. The method of claim 7, wherein the releasing step comprises releasing the nitrogen gas into the atmosphere.

11. The method of claim 7, wherein the brine contains about 12% salt to about 17% salt.

12. The method of claim 7, wherein the halophilic denitrifying microbes are Haloferax mediterranei A4 (NDA4), said denitrifying step comprising removing about 2,300 ppm nitrates from the brine at a rate of 56 ppm/day to 71 ppm/day.

13. A bioremediation device to remove nitrate contaminants from brine, comprising:
an Archaeal bioreactor comprising:
an inlet to fluidly receive the brine therein,
a first outlet to fluidly remove denitrified brine therefrom;
a second outlet through which to release nitrogen gas;
an aqueous medium effective to support anaerobic growth of an Haloarchaea contained within the Archaeal bioreactor; and
a variant of Haloferax mediterranei A1 dispersed within the aqueous medium; and
a brine reservoir fluidly connected to the first outlet.

14. The bioremediation device of claim 13, wherein the variant of Haloferax mediterranei A1 is Haloferax mediterranei (NDA4), said NDA4 growable in brine containing about 7,000 ppm of nitrate.

15. A method for bioremediating nitrate-contaminated brine during a water purification process, comprising:
a) receiving, through the inlet into the Archaeal bioreactor of claim 13, the nitrate-contaminated brine from a spent chromatography column produced during the water purification process;
b) denitrifying the nitrate-contaminated brine via anaerobic respiration by the variant of Haloferax mediterranei A1 thereby producing nitrogen gas;
c) flowing denitrified brine through the first outlet into the brine reservoir;
d) releasing the nitrogen gas through the second outlet into the atmosphere; and
e) regenerating the spent chromatography column with the denitrified brine in the brine reservoir for reuse in the water purification process.

16. The method of claim 15, further comprising repeating steps a) to e) at least once until the water purification process is complete.

17. The method of claim 15, wherein the variant of Haloferax mediterranei A1 is Haloferax mediterranei A4 (NDA4).

18. The method of claim 15, wherein the brine contains about 12% salt to about 17% salt.

19. The method of claim 15, wherein the halophilic denitrifying microbes are Haloferax mediterranei A4 (NDA4), said denitrifying step comprising removing about 2,300 ppm nitrates from the brine at a rate of 56 ppm/day to 71 ppm/day.

20. The method of claim 15, wherein the water in the water purification process is ground water, surface water, wastewater, or well water.
